(19) United States
(12) Patent Application Publication
(10) Pub. No.: US 2020/0093896 A1
(43) Pub. Date: Mar. 26, 2020

(54) Liquid Human Fibrinogen Composition

(71) Applicant: Laboratoire Francais du Fractionnement et des Biotechnologies, Les Ulis (FR)

(72) Inventor: Clemence DEBRAIZE, Les Ulis (FR)

(73) Assignee: Laboratoire Francais du Fractionnement et des Biotechnologies, Les Ulis (KR)

(21) Appl. No.: 16/471,869

(22) PCT Filed: Dec. 22, 2017
(86) PCT No.: PCT/FR2017/053839

§ 371 Date: Jun. 20, 2019

(30) Foreign Application Priority Data
Dec. 22, 2016 (FR) 1663181

(51) Int. Cl. A61K 38/36; A61K 47/18; A61K 47/26

(52) U.S. Cl. CPC A61K 38/363; A61K 9/0019; A61K 47/26; A61K 47/183

(57) ABSTRACT

The invention relates to a pharmaceutical composition comprising between 10 and 30 g/l of fibrinogen, between 10 and 300 mM of arginine and between 10 and 300 mM of glutamate. In addition, the pH of the composition is between 6 and 8.

The invention relates to a human fibrinogen formulation, of use in therapy.

Numerous pathological conditions are currently treated with compositions comprising fibrinogen. Mention may for example be made of hypofibrinogenemia, dysfibrinogenemia or congenital afibrinogenemia in patients with spontaneous or post-traumatic bleeding, supplementary treatment in therapy for uncontrolled severe bleeding in the case of acquired hypofibrinogenemia, etc.

For the treatment of certain pathological conditions, the use of storage-stable and ready-to-use fibrinogen-comprising compositions can prove to be particularly advantageous. This administration form in fact offers practitioners greater flexibility and greater rapidity of administration, improving the urgent treatment of hemorrhagic patients. For this purpose, storage-stable, freeze-dried fibrinogen-comprising compositions suitable for rapid constitution have been developed. However, the reconstitution of such freeze-dried compositions requires a few minutes. Furthermore, the reconstitution must be carried out carefully in order to allow complete dissolution of the freeze-dried material, guaranteeing the concentration of the product, without the formation of foam, or of cloudiness or of deposits that would make the composition difficult or impossible to administer. The use of such freeze-dried products is not therefore optimal in an intra-hospital or peri-hospital emergency medicine context where each minute counts for the treatment of bleeding.

To date, the fibrinogen-comprising compositions are not entirely satisfactory in terms of liquid stability in particular.

In this context, needs for fibrinogen-comprising compositions that are easy to use persist.

### SUMMARY OF THE INVENTION

While working on the problems of stability specific to fibrinogen-comprising compositions, the applicant has developed a specific formulation, combining fibrinogen, arginine and glutamate, in specific ratios contributing to the stability in liquid form of said formulation over time. Surprisingly and advantageously, the applicant has demonstrated that it is possible to obtain fibrinogen-comprising compositions that are particularly stable over time in liquid form, using equivalent amounts of arginine and glutamate. It is not therefore necessary to freeze-dry the fibrinogen-comprising compositions in order to ensure the long-term stability thereof. In addition, the applicant has succeeded in developing fibrinogen-comprising compositions that are particularly suitable for injections, for example intravenous injections, by minimizing the number and the amounts of excipients. The invention makes it possible, generally, to rationalize and simplify the processes for producing these various compositions, resulting in the gaining of time and a reduction in the considerable production costs. Advantageously, said compositions can be free of other excipients, in particular free of excipients known for their freeze-dried product-preserving properties, in order to guarantee stability during storage in ready-to-use liquid form. A subject of the invention is thus a liquid pharmaceutical composition comprising:
between 10 and 30 g/l of fibrinogen;
between 10 and 300 mM of arginine; and
between 10 and 300 mM of glutamate,
   the pH of the composition being between 6 and 8.

In one particular embodiment, the composition consists only of fibrinogen, arginine and glutamate.

The composition according to the invention comprises between 10 g/l and 30 g/l of fibrinogen, preferably between 5 g/l and 25 g/l of fibrinogen, more preferably between 15 g/l and 20 g/l of fibrinogen, and even more preferentially 15 g/l or 20 g/l or 25 g/l of fibrinogen.

According to one preferred embodiment, the fibrinogen is human fibrinogen, in particular obtained from plasma.

The arginine concentration and the glutamate concentration are between 10 and 30 mM, preferentially between 20 and 200 mM, more preferentially between 30 and 100 mM, even more preferentially between 50 and 80 mM.

In one particular embodiment, the arginine concentration and the glutamate concentration are 10-80 mM. Such an arginine and glutamate concentration is particularly suitable for fibrinogen-comprising compositions intended to be injected intravenously.

In one particular embodiment, the arginine concentration is between 10 and 300 mM, preferentially between 20 and 250 mM, more preferentially between 50 and 250 mM.

In another particular embodiment, the arginine concentration is advantageously less than 300 mM, preferably less than 250 mM, preferably less than 200 mM, even more preferentially less than 150 mM. Such an arginine concentration is particularly suitable for avoiding increasing the osmolality by too much.

In one particular embodiment, the glutamate concentration is advantageously less than 80 mM, preferably less than 70 mM, preferably less than 60 mM, even more preferentially less than 50 mM. Such a glutamate concentration is particularly suitable for fibrinogen-comprising compositions intended to be injected intravenously in order to minimize the side effects in patients.

In another advantageous embodiment, the glutamate concentration is between 10 and 80 mM, preferentially between 20 and 70 mM, more preferentially between 30 and 60 mM. Such a glutamate concentration is particularly suitable for fibrinogen-comprising compositions intended to be injected intravenously in order to minimize the side effects in patients.

In one advantageous embodiment, the composition also comprises at least one amino acid chosen from alanine, asparagine, aspartate, cysteine, glutamine, glycine, histidine, isoleucine, leucine, lysine, methionine, phenylalanine, proline, serine, threonine, tryptophan, tyrosine and valine, alone or in combination, preferentially at a concentration of approximately 1-300 mM.

In one particular embodiment, the composition also comprises an amino acid advantageously chosen from serine, proline and/or isoleucine, preferentially at a concentration of approximately 1-300 mM.

In one particular embodiment, the composition also comprises a polar amino acid such as serine, preferentially at a concentration of approximately 1-300 mM.

In one particular embodiment, the composition also comprises a hydrophobic amino acid, such as proline or isoleucine, preferentially at a concentration of approximately 1-300 mM.

In one particular embodiment, the composition also comprises a surfactant, preferentially Pluronic®F68, polysorbate 80 or polysorbate 20 or alkyl sugars, preferentially at a concentration of approximately 1-500 ppm.

In one particular embodiment, the composition also comprises trisodium citrate, preferentially at a concentration of 1-15 mM.

In one particular embodiment, the composition also comprises albumin, at a concentration of 5-1000 ppm, preferentially at a concentration of 5-500 ppm.

The composition according to the invention is advantageously a ready-to-use liquid composition.

The composition according to the invention is advantageously in a form suitable for ocular, nasal, intra-auricular, oral, sublingual, pulmonary, intraperitoneal, intravenous, topical, percutaneous, subcutaneous, intradermal, intramuscular, transdermal, vaginal or rectal administration, preferably intravenous administration.

### DETAILED DESCRIPTION

### Definitions

In the context of the invention, the term “fibrinogen” is intended to mean human fibrinogen. It may be functional human fibrinogen, having a sequence similar or identical to the sequence of normal human fibrinogen, and any intermediate fraction obtained during the process for producing a fibrinogen-comprising composition.

Fibrinogen is a protein consisting of a dimer of three polypeptide chains, called alpha, beta and gamma. Fibrinogen is therefore a dimer and each monomer is composed of three chains (alpha, beta, gamma). The principal form of fibrinogen has a molecular weight (MW) of 340 kDa. Fibrinogen is made up of two identical subunits connected by disulfide bridges, giving the molecule the shape of a fiber comprising three globules: one central (E domain) and two distal (D domains). The whole molecule contains 2964 amino acids: 610 amino acids for the alpha (α) chain, 461 amino acids for the beta (β) chain and 411 amino acids for the gamma (γ) chain. Fibrinogen is involved in primary hemostasis and also in coagulation. It is the most commonly prescribed for the treatment of complications associated with congenital or severe afibrinogenemia and hemorrhagic syndromes or hemorrhagic risks associated with hypofibrinogenemia.

According to the invention, several sources of starting material containing fibrinogen may be used. The fibrinogen-comprising composition according to the invention thus uses a composition of fibrinogen, in particular from various sources. The fibrinogen composition may thus be derived from blood plasma, from cell culture supernatant or from transgenic-animal milk.

In one particular embodiment, the composition according to the invention is a plasma fraction, preferably a plasma fraction obtained from prepurified blood plasma.

The expression “plasma fraction obtained from a prepurified blood plasma” is intended to mean any portion or sub-portion of human blood plasma, having been the subject of one or more purification steps. Said plasma fractions thus include cryosupernatant, plasma cryoprecipitate (resuspension), fraction I obtained by ethanolic fractionation (according to the method of Cohn or of Kistler & Nitschmann), chromatographic eluates and nonadsorbed fractions from chromatography columns, including multicolumn chromatography, and filtrates.

In one preferred embodiment of the invention, the composition according to the invention is derived from a chromatography eluate or from a nonadsorbed fraction from a chromatography column, including multicolumn chromatography.

Thus, in one preferred embodiment of the invention, the composition according to the invention is derived from a plasma fraction obtained from cryosupernatant or resuspended cryoprecipitate.

According to the invention, the “cryosupernatant” corresponds to the liquid phase obtained after thawing of frozen plasma (cryoprecipitation). In particular, the cryosupernatant may be obtained by freezing blood plasma at a temperature of between −10° C. and −40° C., then gentle thawing at a temperature of between 0° C. and +6° C., preferentially between 0° C. and +1° C., followed by centrifugation of the thawed plasma in order to separate the cryoprecipitate and the cryosupernatant. The cryoprecipitate is a concentrate of fibrinogen, fibronectin, von Willebrand factor and factor VIII, whereas the cryosupernatant contains the complement factors, the vitamin K-dependent factors such as protein C, protein S or protein Z, factor II, factor VII, factor IX and factor X, fibrinogen, immunoglobulins and albumin.

In one advantageous embodiment of the invention, the composition according to the invention can be obtained according to the process described by the applicant in application EP 1 739 093 or in application WO 2015/136217.

In another particular embodiment of the invention, the composition according to the invention comes from transgenic-animal milk, for example obtained according to the method described in WO 00/17234 or in WO 00/17239.

In one embodiment, the composition of the invention can be obtained by means of the process comprising the following steps:
an affinity chromatography purification step;
at least one step of rendering biologically safe; and
a step of formulation in liquid form.

In one particular embodiment of the invention, the affinity chromatography purification step is carried out by affinity chromatography using affinity ligands chosen from antibodies, antibody fragments, antibody derivatives or chemical ligands such as peptides, mimetic peptides, peptoids, nanofitins or else oligonucleotide ligands such as aptamers.

In one particular embodiment of the invention, the fibrinogen-comprising stable liquid composition is obtained according to the process comprising the following steps:
obtaining a blood plasma cryosupernatant fraction,
precipitating the cryosupernatant with 8% of ethanol in order to obtain a fibrinogen-enriched fraction,
purifying the fibrinogen-enriched blood plasma fraction, after resuspension, by separation on affinity chromatography gel preferentially using affinity ligands chosen from antibodies, antibody fractions, antibody derivatives or chemical ligands such as peptides, mimetic peptides, peptoids, nanofitins or else oligonucleotide ligands such as aptamers,
recovering the purified adsorbed fraction comprising fibrinogen,
optionally, adding pharmaceutically acceptable excipients.

In one particular embodiment, the process also comprises a step of storage for at least 3 months at 5° C.

In one particular embodiment of the invention, the affinity chromatography used is an affinity matrix with ligands of llama antibody-derived fragment type, such as the Fibrinogen CaptureSelect matrix (Life Technologies).

Particularly advantageously, the composition according to the invention is devoid of proteases and/or of fibrinolysis activators.

The expression “fibrinogen composition devoid of proteases and/or of fibrinolysis activators” is intended to mean that the fibrinogen composition has undergone one or more steps for removing the proteases such as thrombin, prothrombin, plasmin or plasminogen in such a way that the residual amount of proteases and/or of fibrinolysis activators is:
very greatly reduced in comparison to the prepurified fibrinogen solution before the chromatography step, and/or
zero, and/or
less than the detection thresholds of the methods commonly used by those skilled in the art.

Advantageously, the residual prothrombin content is less than 5 μIU/mg of fibrinogen, the plasminogen content is less than 15 ng/mg of fibrinogen.

In one particular embodiment of the invention, the composition according to the invention is thus devoid of proteases such as thrombin and/or plasmin or their corresponding proenzymes prothrombin (coagulation factor II) and/or plasminogen, which are potentially activatable zymogens.

The term “fibrinogen-comprising composition” is intended to mean a composition comprising
human fibrinogen,
optionally one or more copurified or accompanying proteins,
pharmaceutically acceptable excipients.

According to the invention, the copurified or accompanying protein(s) of the fibrinogen may consist of one or more plasma proteins. According to the invention, the term “plasma protein” is intended to mean any protein, and more particularly any protein of industrial or therapeutic interest, contained in the blood plasma. The blood plasma proteins encompass albumin, alpha-macroglobulin, antichymotrypsin, antithrombin, antitrypsin, Apo A, Apo B, Apo C, Apo D, Apo E, Apo F, Apo G, beta XIIa, C1-inhibitor, C-reactive protein, C7, C1r, C1s, C2, C3, C4, C4bP, C5, C6, C1q, C8, C9, carboxypeptidase N, ceruloplasmin, factor B, factor D, factor H, factor I, factor IX, factor V, factor VII, factor VIIa, factor VIII, factor X, factor XI, factor XII, factor XIII, fibrinogen, fibronectin, haptoglobin, hemopexin, heparin cofactor II, histidine-rich GP, IgA, IgD, IgE, IgG, ITI, IgM, kininase II, HMW kininogen, lysozyme, PAI 2, PAI 1, PCI, plasmin, plasmin inhibitor, plasminogen, prealbumin, prekallikrein, properdin, protease nexin INH, protein C, protein S, protein Z, prothrombin, serum amyloid protein (SAP), TFPI, thiol-proteinase, thrombomodulin, tissue factor (TF), TPA, transcobalamin II, transcortin, transferrin, vitronectin, and Willebrand factor. In particular, the plasma proteins encompass the coagulation proteins, that is to say the plasma proteins involved in the cascade reaction chain resulting in the formation of a blood clot. The coagulation proteins encompass factor I (fibrinogen), factor II (prothrombin), factor V (proaccelerin), factor VII (proconvertin), factor VIII (anti-hemophilic factor A), factor IX (anti-hemophilic factor B), factor X (Stuart-Prower factor), factor XI (Rosenthal factor or PTA), factor XII (Hageman factor), factor XIII (fibrin-stabilizing factor or FSF), PK (prekallikrein), HMWK (high molecular weight kininogen), factor III (thromboplastin or tissue factor), heparin cofactor II (HCII), protein C (PC), thrombomodulin (TM), protein S (PS), Willebrand factor (Wf) and tissue factor pathway inhibitor (TFPI), or else tissue factors.

In certain embodiments, the plasma protein consists of a coagulation protein with enzymatic activity. The coagulation proteins with enzymatic activity encompass the activated forms of factor II (prothrombin), factor VII (proconvertin), factor IX (anti-hemophilic factor B), factor X (Stuart-Prower factor), factor XI (Rosenthal factor or PTA), factor XII (Hageman factor), factor XIII (fibrin-stabilizing factor or FSF) and PK (prekallikrein).

The term “pharmaceutically acceptable excipient” corresponds to any excipient that can be advantageously used for the formulation of human proteins, in particular to substances chosen from salts, amino acids, sugars, surfactants or any other excipient.

The term “equimolar” refers to an identical or equivalent amount of moles/1 or mmoles/1 (M or mM) between several excipients, in particular between two excipients, at a ratio between the two excipients of between 0.8 and 1.2, preferentially between 0.9 and 1.1, even more preferentially approximately equal to 1.0. In particular, the composition according to the invention advantageously comprises an equimolar amount of arginine and glutamate, with an arginine/glutamate ratio of between 0.8 and 1.2, preferentially between 0.9 and 1.1, even more preferentially approximately equal to 1.0; or a glutamate/arginine ratio of between 0.8 and 1.2, preferentially between 0.9 and 1.1, and even more preferentially approximately equal to 1.0.

The term “stable” corresponds to the physical and/or chemical stability of the fibrinogen-comprising composition. The term “physical stability” refers to the reduction or absence of formation of insoluble or soluble aggregates of the dimeric, oligomeric or polymeric forms of fibrinogen, to the reduction or absence of the formation of precipitate, and also to the reduction or absence of any structural denaturation of the molecule.

The term “chemical stability” refers to the reduction or absence of any chemical modification of the fibrinogen-comprising composition during storage, in liquid state, under accelerated conditions.

The stability of a fibrinogen-comprising composition can be evaluated by various methods, in particular by accelerated stability test methods or by long-term stability testing methods.

The accelerated stability test methods comprise in particular mechanical stress tests by heating.

In one embodiment, the stability of the fibrinogen-comprising composition is measured after a heat stress by heating in a thermostatic bath at 37° C., for example by measuring at T0, T+7 days and T+14 days. The particles in solution having sizes greater than the threshold of detection by the human eye (approximately 50 μm) are then measured in particular by visual inspection using for example a European pharmacopeia inspecting device (opalescence, particle formation), by measuring the turbidity by means of a spectrophotometer measuring absorbance or optical density at 400 nm.

A long-term stability test can be carried out under various temperature, humidity and light conditions. Preferentially, in the context of the present invention, the stability test can last a minimum of 1 week, preferentially at least 1 month, preferentially at least 2 months, preferentially at least 3 months, preferentially at least 4 months, preferentially at least 5 months, more preferentially at least 6 months. Typically, the measurement of the stability parameters, as defined below, takes place
before subjecting a fibrinogen-comprising composition to stability testing; reference is then made to initial content; and
during or at the end of said stability test, it being understood that said stability test can last a minimum of 1 week, preferentially at least 1 month, preferentially at least 2 months, preferentially at least 3 months, preferentially at least 4 months, preferentially at least 5 months, more preferentially at least 6 months.

The methods of analysis after subjecting to long-term stability comprise in particular analyses by visual inspection using in particular a European pharmacopeia inspecting device (opalescence, particle formation), by measuring the turbidity by means of a spectrophotometer measuring absorbance or optical density at 400 nm, making it possible to evaluate the presence or absence of degradation of the product by detecting cloudiness in the solution.

In one embodiment, the stability of the fibrinogen-comprising composition is defined by the measurement of the content of monomers preserved during the stability test by means of the High Pressure Size Exclusion Chromatography (HPSEC) method. These methods are well known to those skilled in the art.

A fibrinogen composition is advantageously considered to be stable if the amount of fibrinogen monomers preserved during the subjecting to stability testing is greater than 50%, preferentially greater than 60%, preferentially greater than 70%, preferentially greater than 80%, preferentially greater than 90%, preferentially greater than 95% of the initial content of fibrinogen monomers.

More preferentially, the amount of fibrinogen monomers preserved during the stability test is greater than 70% of the initial content of fibrinogen monomers.

The expression “initial content of fibrinogen monomer” is intended to mean the content of monomer observed before the subjecting to stability testing. Typically, the amount of fibrinogen monomer is measured before the subjecting to stability testing and during or at the end of said stability test.

Alternatively, a fibrinogen composition is considered to be stable if the variation in amount of fibrinogen monomers during the stability test is less than 20%, preferentially less than 10%, preferentially less than 5%, preferentially less than 1%.

In another embodiment, the stability of the fibrinogen-comprising composition is defined by the measurement of the content of fibrinogen polymers formed during the subjecting to stability testing by means of HPSEC. The fibrinogen polymers are polymers comprising at least 2 alpha polypeptide chains, 2 beta polypeptide chains and 2 gamma polypeptide chains of fibrinogen. This term also includes the fibrinogen trimers.

A fibrinogen composition is advantageously considered to be stable if the amount of fibrinogen polymers formed during the subjecting to stability testing is less than 10%, preferentially less than 20%, preferentially less than 30%, preferentially less than 40%, preferentially less than 50% relative to the initial content of fibrinogen polymers. Typically, the initial content of fibrinogen polymers corresponds to all of the polymeric forms (timers and more) of fibrinogen before the subjecting to stability testing.

More preferentially, the amount of fibrinogen polymers formed during the subjecting to stability testing is less than 30%. Typically, the amount of fibrinogen polymers is measured before the subjecting to stability testing and during or at the end of said stability test.

Alternatively, a fibrinogen composition is considered to be stable if the variation in amount of fibrinogen polymers during the stability test is less than 20%, preferentially less than 10%, preferentially less than 5%, preferentially less than 1%.

In another embodiment, the stability of the fibrinogen-comprising composition is evaluated by the measurement of the coagulable activity of fibrinogen relative to the antigenic measurement of fibrinogen (also called specific activity). Particularly advantageously, the stable fibrinogen composition has a coagulable fibrinogen/antigenic fibrinogen ratio of greater than 0.5; preferentially greater than 0.6; greater than 0.7; greater than 0.8; greater than 0.9; even more preferentially approximately equal to 1.0.

The term “coagulable fibrinogen” is intended to mean the measurement of the functional fibrinogen by a coagulation technique, determined according to the method of von Clauss. The coagulable activity is expressed in g/1 of fibrinogen solution. This technique is known to those skilled in the art who may refer to the publication Von Clauss, A. (1957) Gerinnungsphysiologische schnellmethode zur bestimmung des fibrogens. Acta Haematologica, 17, 237-246.

The term “antigenic fibrinogen” is intended to mean the amount of fibrinogen, whether active or nonactive, measured by the nephelometric method. The amount of antigenic fibrinogen is expressed in g/l.

The stability of the fibrinogen-comprising composition is also evaluated by the SDS PAGE measurement of the preservation of the alpha, beta and gamma fibrinogen chains, preferentially before and after a stability test as defined in the context of the present invention. Thus, a fibrinogen composition is advantageously considered to be stable if:
all of the alpha chains are at least 50%, preferentially at least 60%, preferentially at least 70%, preferentially at least 80%, preferentially at least 90% preserved; more preferentially approximately 100% preserved, and/or
all of the beta chains are at least 50%, preferentially at least 60%, preferentially at least 70%, preferentially at least 80%, preferentially at least 90% preserved; more preferentially approximately 100% preserved, and/or
all of the gamma chains are at least 50%, preferentially at least 60%, preferentially at least 70%, preferentially at least 80%, preferentially at least 90% preserved; more preferentially approximately 100% preserved.

The stability of the fibrinogen-comprising composition is also defined by the measurement of the turbidity by means of UV spectrophotometry at 400 nm. Specifically, the turbidity reflects the amount of material which makes the solution cloudy. A fibrinogen composition is advantageously considered to be stable if the turbidity measured after the stability test as defined in the present invention is comparable to the turbidity measured before stability.

Advantageously, the turbidity measured after the subjecting to stability testing corresponds to less than 130%, less than 120%, less than 110%; advantageously corresponds to 100% of the turbidity measured before stability.

In one advantageous embodiment of the invention, the fibrinogen-comprising composition is stable for at least 1 month, at least 2 months, at least 3 months, at least 4 months, at least 5 months, at least 6 months at between 2° C. and 8° C.

In one advantageous embodiment of the invention, the fibrinogen-comprising composition is stable for 6 months between 2° C. and 8° C.

The expression “fibrinogen composition in liquid form” is intended to mean a composition comprising fibrinogen in solution, preferably which has not been subjected to a freeze-drying, desiccation, dehydration, spray-drying or drying step, and which does not therefore need to be reconstituted before use.

In the context of the invention, the expression “between x and y” means that the values x and y are included.

### Formulations

The composition according to the invention comprises between 10 g/l and 30 g/l of fibrinogen (referenced below 10-30 g/l fibrinogen compositions), preferably between 15 g/l and 25 g/l of fibrinogen, more preferably between 15 g/l and 20 g/l of fibrinogen, and even more preferentially 15 g/l or 20 g/l or 25 g/l of fibrinogen.

In the context of the present invention, the concentrations are meant in terms of the ready-to-use final composition. The concentrations are determined with respect to the compositions in liquid form.

Particularly advantageously, the applicant has demonstrated that it is possible to obtain liquid compositions comprising 10-30 g/l of fibrinogen that are particularly stable over time using a minimum of excipients. Thus, according to the invention, the 10-30 g/l liquid fibrinogen compositions advantageously comprise between 10 and 300 mM of arginine, preferably between 20 and 200 mM, and between 10 and 300 mM of glutamate, preferably between 20 and 200 mM.

In one particular embodiment, the arginine concentration is between 10 and 300 mM, preferentially between 20 and 250 mM, more preferentially between 50 and 250 mM.

In another particular embodiment, the arginine concentration is advantageously less than 300 mM, preferably less than 250 mM, preferably less than 200 mM, even more preferentially less than 150 mM. Such an arginine concentration is particularly suitable for avoiding increasing the osmolality by too much.

In one particular embodiment, the glutamate concentration is advantageously less than 80 mM, preferably less than 70 mM, preferably less than 60 mM, even more preferentially less than 50 mM. Such a glutamate concentration is particularly suitable for the fibrinogen-comprising compositions intended to be injected intravenously in order to minimize the side effects in patients.

In another advantageous embodiment, the glutamate concentration is between 10 and 80 mM, preferentially between 20 and 70 mM, more preferentially between 30 and 60 mM. Such a glutamate concentration is particularly suitable for the fibrinogen-comprising compositions intended to be injected intravenously in order to minimize the side effects in patients.

In another particular embodiment, the 10-30 g/l liquid fibrinogen composition comprises arginine and glutamate in equimolar amounts. The applicant has demonstrated, surprisingly, that the addition of equimolar amounts of glutamate and arginine advantageously makes it possible to stabilize the formulation in liquid form while at the same time maintaining good tolerance with respect to patients. The stabilization effect is proportional to the increase in the amounts of glutamate and arginine. The applicant has nevertheless demonstrated an optimum effect between 10 and 80 mM; indeed, above 80 mM, the composition, although stable, has a glutamate content capable of causing side effects in patients.

Likewise, in one particular embodiment, the 10-30 g/l fibrinogen compositions advantageously comprise between 1 and 500 ppm of surfactant, preferentially between 20 and 400 ppm of surfactant, more preferentially between and 400 ppm of surfactant, more preferentially between 150 and 250 ppm. The surfactant used in the composition according to the invention is advantageously chosen from nonionic surfactants, preferably polysorbates, and in particular from polysorbate 80 (or Tween®80 which is polyoxyethylenesorbitan monooleate) and polysorbate 20 (or Tween®20 which is polyoxyethylenesorbitan monolaurate). Optionally, the surfactant can be chosen from poloxamers, polyoxyethylene alkyl ethers, an ethylene/polypropylene copolymer block, alkyl glucosides or alkyl sugars, and Pluronic®F68 (polyethylene polypropylene glycol). The surfactants can also be combined with one another. Advantageously, the surfactant makes it possible to stabilize the interactions between the molecules in liquid form.

According to one particular embodiment of the invention, the composition also comprises at least one amino acid chosen from alanine, asparagine, aspartate, cysteine, glutamine, glycine, histidine, isoleucine, leucine, lysine, methionine, phenylalanine, proline, serine, threonine, tryptophan, tyrosine and valine, alone or in combination, preferentially at a concentration of approximately 1-300 mM.

According to one particular embodiment of the invention, the composition also comprises an amino acid advantageously chosen from serine, proline and/or isoleucine, preferentially at a concentration of approximately 1-300 mM. Advantageously, the amino acid stabilizes the composition according to the invention in liquid form.

In one particular embodiment, the composition also comprises a polar amino acid such as serine, preferentially at a concentration of approximately 1-300 mM.

According to one particular embodiment of the invention, the composition also contains at least one hydrophobic amino acid chosen from leucine, alanine, phenylalanine, tryptophan, valine, methionine, isoleucine, proline, cysteine and/or glycine. Preferably, the hydrophobic amino acid is chosen from proline and/or isoleucine. Advantageously, the hydrophobic amino acid stabilizes the composition according to the invention in liquid form.

According to one particular embodiment of the invention, the composition also contains a buffer, in particular a histidine buffer, a phosphate buffer, Tris-HCl or trisodium citrate, preferentially trisodium citrate, preferentially at a concentration of between 1 and 15 mM, more preferentially at a concentration of between 7 and 10 mM, even more preferentially at a concentration of between 8 and 9 mM.

According to one particular embodiment of the invention, the composition also contains albumin at a concentration of between 5 and 1000 ppm, preferentially at a concentration of between 5 and 500 ppm. Advantageously, the albumin present in the composition of the invention is human plasma albumin or recombinant albumin.

Advantageously, the applicant has demonstrated that such compositions have an osmolality particularly suitable for administration by injection, in particular intravenous injection, this being without the addition of excipients, in terms of number and/or amounts. Thus, the invention provides 15-25 g/l or 15-20 g/l fibrinogen compositions having a measured osmolality of between 250 and 550 mOsm/kg approximately. In the context of the invention, and unless otherwise mentioned, the osmolality of the composition means the osmolality measured in said composition.

The osmolality is advantageously measured using an osmometer calibrated with standard solutions, and in particular according to the method specified by the European pharmacopeia (European pharmacopeia 5.0 of 2005-01/2005:2.2.35.). Of course, any other method for measuring osmolality may be used.

In one particular embodiment, the only excipients of the fibrinogen composition according to the invention are arginine and glutamate. Such a formulation allows good stabilization of the liquid fibrinogen compositions and a reduction in the industrial-scale production times and costs by virtue of the presence of an effective minimum number and amount of excipients. Advantageously, such a composition has an osmolality compatible with administration by injection, in particular intravenous injection.

In another particular embodiment, the only excipients of the fibrinogen composition according to the invention are arginine, glutamate and the surfactant.

In another particular embodiment, the only excipients of the fibrinogen composition according to the invention are arginine, glutamate and at least one other amino acid.

In another particular embodiment, the only excipients of the fibrinogen composition according to the invention are arginine, glutamate and at least one hydrophobic amino acid.

In another particular embodiment, the only excipients of the fibrinogen composition according to the invention are arginine, glutamate, the surfactant and at least one other amino acid.

In another particular embodiment, the only excipients of the fibrinogen composition according to the invention are arginine, glutamate, the surfactant and at least one hydrophobic amino acid.

In another particular embodiment, the only excipients of the fibrinogen composition according to the invention are arginine, glutamate and human albumin.

In one particular embodiment, the composition consists essentially of fibrinogen, arginine and glutamate, in the sense that any other excipient that might be present would only be so in trace amounts.

In another particular embodiment of the invention, the composition according to the invention is devoid of divalent ions, in particular divalent metal ions, in particular calcium and/or sodium ions.

Particularly advantageously, the composition according to the invention is devoid of excipients known for their freeze-dried material-stabilizing role, such as sodium chloride and/or carboxymethyldextran (CMD).

In another particular embodiment of the invention, the composition according to the invention is devoid of albumin.

In another particular embodiment of the invention, the composition according to the invention is devoid of sugars.

In one particular embodiment of the invention, the fibrinogen composition according to the invention is devoid of protease inhibitors and/or of anti-fibrinolytics.

The expression “protease inhibitors and/or anti-fibrinolytics” is intended to mean any molecule with antiprotease activity, in particular any molecule with serine-protease-inhibiting and/or anti-fibrinolytic activity, in particular any molecule with thrombin-inhibiting and/or anti-plasmin activity, in particular hirudin, benzamidine, aprotinin, phenylmethylsulfonyl fluoride (PMSF), pepstatin, leupeptin, antithrombin III optionally combined with heparin, alpha-2-macroglobulin, alpha-1 antitrypsin, hexanoic or epsilon-aminocaproic acid, tranexamic acid, alpha2-antiplasmin, diisopropylfluorophosphate (DSP), antichimotrypsin.

In one particular embodiment of the invention, the fibrinogen composition according to the invention is devoid of hirudin and/or of benzamidine and/or of aprotinin and/or of PMSF and/or pepstatin and/or of leupeptin and/or of antithrombin III optionally combined with heparin and/or of alpha-2-macroglobulin and/or of alpha-1 antitrypsin and/or of hexanoic and/or epsilon-aminocaproic acid and/or of tranexamic acid and/or of alpha2-antiplasmin.

In one particular embodiment of the invention, the composition according to the invention does not comprise other copurified proteins, advantageously no FXIII and/or no fibronectin.

In another particular embodiment of the invention, the fibrinogen composition according to the invention may also comprise one or more accompanying, optionally copurified, proteins. In one particular embodiment of the invention, the composition according to the invention advantageously comprises FXIII.

Advantageously, the composition according to the invention is devoid of fibrin.

In another particular embodiment, the composition consists essentially of fibrinogen, arginine, glutamate, optionally a surfactant, preferably tween 80, optionally hydrophobic amino acid, preferentially proline and/or isoleucine, and optionally buffer such as trisodium citrate, in the sense that any other excipient that might be present would only be so in trace amounts.

According to the invention, the final pH of the composition is advantageously between 6 and 8. Preferentially, the pH is approximately 6.0-7.5, even more preferentially between 6.0 and 7.0. A pH of 6.0-7.0 in fact gives particularly satisfactory results in terms of liquid stability over time while at the same time making it possible to limit both the aggregation phenomenon and the degradation phenomenon. The final pH means the pH of the composition after formulation, that is to say in the ready-to-use composition. Unless otherwise mentioned, in the present description, the pH of the composition denotes the final pH.

A preferred 10-30 g/l fibrinogen composition according to the invention comprises:
10-300 mM of arginine
10-300 mM of glutamate
optionally 1-15 mM of buffer, in particular of trisodium citrate
optionally 1-300 mM of another amino acid, in particular 1-300 mM of proline and/or 1-300 mM of isoleucine and/or 1-300 mM of serine
optionally 1-500 ppm of surfactant, preferentially 20-400 ppm, more preferably 150-250 ppm
optionally 5-1000 ppm of human albumin
   the pH of the composition being 6.0-8.0.

A preferred 10-30 g/l fibrinogen composition according to the invention comprises:
20-200 mM of arginine
20-200 mM of glutamate
optionally 7-10 mM of buffer, in particular of trisodium citrate
optionally 1-100 mM of another amino acid, in particular 1-100 mM of proline and/or 1-100 mM of isoleucine and/or 1-100 mM of serine
optionally 20-400 ppm of surfactant
optionally 5-500 ppm of human albumin
   the pH of the composition being 6.0-8.0.

A preferred 15-25 g/l fibrinogen composition according to the invention comprises:
150 mM of arginine
80 mM of glutamate
8.5 mM of buffer, in particular of trisodium citrate
200 ppm of surfactant, preferentially of polysorbate80, of polysorbate20 or of Pluronic®F68
optionally 1-100 mM of another amino acid, in particular 1-100 mM of proline and/or 1-100 mM of isoleucine and/or 1-100 mM of serine
optionally 5-500 ppm of human albumin
   the pH of the composition being 6.0-7.0.

A preferred 15-25 g/l fibrinogen composition according to the invention comprises:
10-300 mM of arginine
10-300 mM of glutamate
   the pH of the composition being 6.0-8.0.

A preferred 15-20 g/l fibrinogen composition according to the invention comprises:
10-300 mM of arginine
10-300 mM of glutamate
   the pH of the composition being 6.0-8.0.

Another preferred 15-25 g/l fibrinogen composition according to the invention comprises:
10-300 mM of arginine
10-300 mM of glutamate
1-15 mM of buffer, in particular of trisodium citrate
   the pH of the composition being 6.0-8.0.

Another preferred 15-20 g/l fibrinogen composition according to the invention comprises:
10-300 mM of arginine
10-300 mM of glutamate
1-15 mM of trisodium citrate
   the pH of the composition being 6.0-8.0.

Another preferred 15-25 g/l fibrinogen composition according to the invention comprises:
10-300 mM of arginine
10-300 mM of glutamate
1-300 mM of another amino acid
   the pH of the composition being 6.0-8.0.

Another preferred 15-20 g/l fibrinogen composition according to the invention comprises:
10-300 mM of arginine
10-300 mM of glutamate
1-300 mM of hydrophobic amino acid
   the pH of the composition being 6.0-8.0.

Another preferred 15-25 g/l fibrinogen composition according to the invention comprises:
10-300 mM of arginine
10-300 mM of glutamate
1-300 mM of another amino acid
1-15 mM of buffer, in particular of trisodium citrate
   the pH of the composition being 6.0-8.0.

Another preferred 15-20 g/l fibrinogen composition according to the invention comprises:
10-300 mM of arginine
10-300 mM of glutamate
1-300 mM of hydrophobic amino acid
1-15 mM of trisodium citrate
   the pH of the composition being 6.0-8.0.

Another preferred 15-25 g/l fibrinogen composition according to the invention comprises:
10-300 mM of arginine
10-300 mM of glutamate
1-300 mM of proline and/or of isoleucine and/or of serine
   the pH of the composition being 6.0-8.0.

Another preferred 15-20 g/l fibrinogen composition according to the invention comprises:
10-300 mM of arginine
10-300 mM of glutamate
1-300 mM of proline and/or of isoleucine and/or of serine
   the pH of the composition being 6.0-8.0.

Another preferred 15-20 g/l fibrinogen composition according to the invention comprises:
10-300 mM of arginine
10-300 mM of glutamate
1-300 mM of proline and/or of isoleucine
   the pH of the composition being 6.0-8.0.

Another preferred 15-20 g/l fibrinogen composition according to the invention comprises:
10-300 mM of arginine
10-300 mM of glutamate
1-500 ppm of surfactant, preferentially 50-400 ppm, more preferably 150-250 ppm the pH of the composition being 6.0-8.0.

Another preferred 15-20 g/l fibrinogen composition according to the invention comprises:
10-300 mM of arginine
10-300 mM of glutamate
1-500 ppm of surfactant, preferentially 50-400 ppm, more preferably 150-250 ppm
1-15 mM of trisodium citrate
   the pH of the composition being 6.0-8.0.

Another preferred 15-20 g/l fibrinogen composition according to the invention comprises:
10-300 mM of arginine
10-300 mM of glutamate
1-500 ppm of polysorbate 80, preferentially 50-400 ppm, more preferably 150-250 ppm
   the pH of the composition being 6.0-8.0.

Another preferred 15-20 g/l fibrinogen composition according to the invention comprises:
10-300 mM of arginine
10-300 mM of glutamate
1-300 mM of hydrophobic amino acid
1-500 ppm of supernatant, preferentially 50-400 ppm, more preferably 150-250 ppm
1-15 mM of trisodium citrate
   the pH of the composition being 6.0-8.0.

Another preferred 15-20 g/l fibrinogen composition according to the invention comprises:
10-300 mM of arginine
10-300 mM of glutamate
1-300 mM of proline and/or of isoleucine
1-500 ppm of polysorbate 80, preferentially 50-400 ppm, more preferably 150-250 ppm
optionally 1-15 mM of trisodium citrate
   the pH of the composition being 6.0-8.0.

The measured osmolality of this fibrinogen composition is advantageously approximately 225-715 mOsm/kg.

Surprisingly and advantageously, the applicant has demonstrated that an arginine concentration of approximately 10-80 mM, +/−10%, combined with a glutamate concentration of approximately 10-80 mM, +/−10%, is sufficient to keep the liquid 15-20 g/l fibrinogen composition stable while at the same time maintaining an osmolality of between 225 and 500 mOsm/kg in the composition, although higher concentrations would have been expected in order to guarantee the liquid stability, increasing in parallel the osmolality of said compositions. In point of fact, an excessively high osmolality can be responsible for cell dehydration (exiting of intracellular water to the extracellular medium) prejudicial to the patient.

Advantageously, the composition according to the invention has a purity greater than or equal to 70%, preferably greater than or equal to 75%, 80%, 85%, 90%, 95%, 96%, 97%, 98%, 99%.

The fibrinogen composition according to the invention, in liquid form and after storage for at least a period of 6 months between 2° C. and 8° C., advantageously has an amount of fibrinogen monomers preserved during the subjecting to stability testing of greater than 50%, preferentially greater than 60%, preferentially greater than 70%, preferentially greater than 80%, preferentially greater than 90%, preferentially greater than 95% of the initial content of fibrinogen monomers.

More preferentially, the fibrinogen composition according to the invention, in liquid form and after storage for at least a period of 6 months at 5° C.±3° C., advantageously has an amount of fibrinogen monomers preserved during the subjecting to stability testing of greater than 70% of the initial content of fibrinogen monomers.

Alternatively, the fibrinogen composition according to the invention, in liquid form, advantageously has a variation in amount of fibrinogen monomers during the stability test of less than 20%, preferentially less than 10%, preferentially less than 5%, preferentially less than 1%.

The fibrinogen composition according to the invention, in liquid form and after storage for at least a period of 6 months between 2° C. and 8° C., advantageously has an amount of fibrinogen polymers formed during the subjecting to stability testing of less than 10%, preferentially less than 20%, preferentially less than 30%, preferentially less than 40%, preferentially less than 50%, relative to the initial content of fibrinogen polymers. Typically, the initial content of fibrinogen polymers corresponds to all of the polymeric forms (trimers and higher) of fibrinogen before the subjecting to stability testing.

More preferentially, the fibrinogen composition according to the invention, in liquid form and after storage for at least a period of 6 months between 2° C. and 8° C., advantageously has an amount of fibrinogen polymers formed during the subjecting to stability testing of less than 30%. Typically, the amount of fibrinogen polymers is measured before the subjecting to stability testing and during or at the end of said stability test.

Alternatively, the fibrinogen composition according to the invention, in liquid form, advantageously has a variation in amount of fibrinogen polymers during the stability test of less than 20%, preferentially less than 10%, preferentially less than 5%, preferentially less than 1%.

The fibrinogen composition according to the invention, in liquid form and after storage for at least a period of 6 months between 2° C. and 8° C., advantageously has a coagulable fibrinogen/antigenic fibrinogen ratio of greater than 0.5; preferentially greater than 0.6; greater than 0.7; greater than 0.8; greater than 0.9; even more preferentially approximately equal to 1.0.

The fibrinogen composition according to the invention, in liquid form and after storage for at least a period of 6 months between 2° C. and 8° C., advantageously exhibits
an at least 50%, preferentially at least 60%, preferentially at least 70%, preferentially at least 80%, preferentially at least 90% preservation of all of the alpha chains; more preferentially approximately 100% preserved, and/or
an at least 50%, preferentially at least 60%, preferentially at least 70%, preferentially at least 80%, preferentially at least 90% preservation of all of the beta chains; more preferentially approximately 100% preserved, and/or
an at least 50%, preferentially at least 60%, preferentially at least 70%, preferentially at least 80%, preferentially at least 90% preservation of all of the gamma chains; more preferentially approximately 100% preserved.

The fibrinogen composition according to the invention, in liquid form and after storage for at least a period of 6 months between 2° C. and 8° C., advantageously exhibits a turbidity, measured after the subjecting to stability testing, corresponding to less than 130%, less than 120%, less than 110%; advantageously corresponding to 100% of the turbidity measured before stability.

The compositions of the invention may be pharmaceutical compositions, that is to say compositions suitable for therapeutic use. The pharmaceutical compositions of the invention are thus of use as medicaments, in particular in order to treat hypofibrinogenemia, dysfibrinogenemia or congenital afibrinogenemia in patients presenting spontaneous or post-traumatic bleeding, or as a supplementary treatment in the therapy of uncontrolled severe bleeding in the context of acquired hypofibrinogenemia.

According to the invention, use may be made of several sources of starting material containing fibrinogen. The fibrinogen composition may thus be derived from blood plasma, otherwise known as plasma fractions, from cell culture supernatant or from transgenic animal milk.

In one preferred embodiment, the composition of the invention has undergone no prior freeze-drying, desiccation, dehydration or drying step.

In one preferred embodiment, the composition of the invention has undergone no prior freeze-dried product reconstitution step.

The compositions according to the invention may advantageously be subjected to at least one infectious agent removal or inactivation method.

A viral inactivation often comprises a treatment with chemical products, for example with solvent and/or detergent and/or with heat (pasteurization and/or heating) and/or with irradiation (gamma and/or UVC irradiation) and/or by pH treatment (treatment at acid pH).

Preferably, the viral inactivation consists of a step of treatment by heating or by treatment with solvent and detergent. The treatment with solvent and detergent (generally referred to as solvent/detergent or S/D treatment) comprises in particular treatment with tri-n-butyl phosphate (TnBP) and/or a detergent which is chosen from Triton X-100, Tween (preferably Tween 80), sodium cholate and 2-[4-(2,4,4-trimethylpentan-2-yl)phenoxy]ethanol (Octoxinol).

Preferably, the viral removal step consists of a nanofiltration which can be used to remove the infectious agents, in particular viruses and UTAs. In the case of plasma proteins, nanofiltration generally refers to the filtration of the concentrate of proteins of interest through a filter with a pore size of less than 80 nm. The filters available are for example the Planova BioEx, Planova® 75N, Planova® 35N, Planova® 20N or Planova® 15N (Asahi corporation), Pegasus SV4, Ultipor DV 50 or DV 20 (Pall corporation), Virosart CPV, Virosart HC or Virosart HF (Sartorius), Viresolve® NFR, Pro or NFP (Millipore) filters. The nanofiltration can advantageously be carried out on a single filter or on several filters in series having an identical or decreasing porosity.

The removal of the infectious agents can also be carried out by means of depth filtration. The filters available are, for example, filters composed of regenerated cellulose, to which filtration adjuvants may have been added (such as cellite, pearlite or Kieselguhr earth) sold by Cuno (Zeta+VR series filters), Pall-Seitz (P-series Depth Filter) or Sartorius (Virosart CPV, Sartoclear P depth filters).

After purification and at least one infectious agent removal or inactivation step, the composition according to the invention is directly subjected to the steps of pharmaceutical forming in liquid form: formulation, sterilizing filtration and dispensing into a container (bottle or other storage/administration device).

Particularly advantageously, the composition according to the invention is subjected to no freeze-drying, desiccation, dehydration or drying step.

Particularly advantageously, the composition according to the invention is thus in liquid form without having been subjected to a step for reconstituting a freeze-dried product.

The following examples illustrate the invention without limiting the scope thereof.

### EXAMPLES

### Example 1: Stable Liquid Fibrinogen Composition

The fibrinogen is obtained according to the method described in patent FR 0 506 640.

The final product is then dyalized against an 8.5 mM trisodium citrate dihydrate buffer: (0.175 mM of citric acid+8.325 mM sodium citrate) at pH 7.0±0.2 in order to obtain a deformulated fibrinogen.

### Example 2: Stable Liquid Fibrinogen Composition

Materials & Methods

Plasma Fraction

The starting material is a pool of human plasma subjected beforehand to a cryoprecipitation step then subjected to a step of precipitation with 8% ethanol.

The prepurified plasma fibrinogen solution thus obtained is diluted with 2 volumes of gel equilibration buffer before injection onto the chromatography column pre-equilibrated with respect to pH and conductivity.

Characteristics of the plasma fraction: antigenic fibrinogen adjusted by dilution to 4.7-4.8 g/l.

Buffer Solutions

The composition of the buffer solutions used during the various steps of the chromatography process is summarized in table 1 below.

TABLE 1
Buffer solutions for affinity chromatography

| Phases | Compositions | Target values |
|---|---|---|
| Column | 10 mM trisodium citrate, | pH adjusted to 7.4 |
| equilibration and | 0.1M arginine HCl | |
| return to baseline | | |
| Pre-elution | 10 mM trisodium citrate, | pH adjusted to 7.4 |
| | 2.0M sodium chloride | |
| | 0.1M arginine HCl | |
| Elution | 10 mM trisodium citrate, | pH adjusted to 7.4 |
| | 50% v/v propylene glycol | |
| | 1.0M arginine HCl | |

Affinity Chromatography Gels

For the chromatography, a CaptureSelect Fibrinogen affinity gel (Life Technologies ref. 191291050, batches 171013-01 and 171013-05) are used.

Columns

A column 50 mm in diameter (reference XK 50/30 GE Healthcare), packed gel volume of 67 ml; for column height of 3.4 cm was used.

Affinity Purification

The fibrinogen solution adjusted to approximately 5 g/l is injected without other adjustment onto the equilibrated CaptureSelect Fibrinogen affinity column. A load of approximately 10 g/l was applied.

The chromatography eluate was then ultrafiltered and preformulated on a membrane with a cut-off threshold of 100 kDa (reference Pall Omega OS100C10).

At the end of the process, the product obtained has a coagulable fibrinogen concentration of 15.2 g/l and an antigenic fibrinogen concentration of 15.2 mg/ml.

### Example 3: Stability of a Liquid Fibrinogen Composition by Accelerated Stability Test

Fibrinogen Compositions

The composition obtained in example 1 or in example 2 is formulated with various excipients.

The formulations are reproduced in table 2 below:

| | | | | Amino | Trisodium | | | |
|---|---|---|---|---|---|---|---|---|
| | | Glutamate | Arginine | acid | citrate | | | |
| Formulation | Fibrinogen | (mM) | (mM) | (mM) | (mM) | Surfactant | pH | Osmolality |
| F1 | Derived from | 50 | 50 | | 8.5 | Polysorbate | 7.0 | |
| | example 1 | | | | | 80 - 200 ppm | | |
| F2 | Derived from | 150 | 150 | | 8.5 | Polysorbate | 7.0 | 564 |
| | example 1 | | | | | 80 - 200 ppm | | |
| F3 | Derived from | 175 | 175 | | 8.5 | Polysorbate | 7.0 | |
| | example 1 | | | | | 80 - 200 ppm | | |
| F4 | Derived from | 200 | 200 | | 8.5 | Polysorbate | 7.0 | |
| | example 1 | | | | | 80 - 200 ppm | | |
| F5 | Derived from | 300 | 300 | | 8.5 | Polysorbate | 7.0 | |
| | example 1 | | | | | 80 - 200 ppm | | |
| F6 | Derived from | 60 | 150 | | 8.5 | Polysorbate | 7.0 | 403 |
| | example 1 | | | | | 80 - 200 ppm | | |
| F7 | Derived from | 70 | 150 | | 8.5 | Polysorbate | 7.0 | 423 |
| | example 1 | | | | | 80 - 200 ppm | | |
| F8 | Derived from | 80 | 150 | | 8.5 | Polysorbate | 7.0 | 438 |
| | example 1 | | | | | 80 - 200 ppm | | |
| F9 | Derived from | 80 | 135 | | 8.5 | Polysorbate | 7.0 | 414 |
| | example 1 | | | | | 80 - 200 ppm | | |
| F10 | Derived from | 80 | 120 | | 8.5 | Polysorbate | 7.0 | 387 |
| | example 1 | | | | | 80 - 200 ppm | | |
| F11 | Derived from | 80 | 80 | | 8.5 | Polysorbate | 7.0 | |
| | example 1 | | | | | 80 - 200 ppm | | |
| F12 | Derived from | 70 | 70 | | 8.5 | Polysorbate | 7.0 | |
| | example 1 | | | | | 80 - 200 ppm | | |
| F13 | Derived from | 60 | 60 | | 8.5 | Polysorbate | 7.0 | |
| | example 1 | | | | | 80 - 200 ppm | | |
| F14 | Derived from | 50 | 100 | Proline: 60 | 8.5 | Polysorbate | 7.0 | |
| | example 1 | | | Glycine: 27 | | 80 - 200 ppm | | |
| F15 | Derived from | 50 | 100 | Proline: 60 | 8.5 | Polysorbate | 7.0 | |
| | example 2 | | | Glycine: 27 | | 80 - 200 ppm | | |

Analysis Protocols

The compositions are subjected to a heating stress by heating in a thermostatic chamber at 37° C., then removed from the thermostatic chamber for analysis at T0, T+7 days and T+14 days.

The various analyses carried out are the following:
Visual inspection: opalescence and formation of visible particles are determined by visual inspection carried out with a European pharmacopeia inspecting device.
Turbidity.
Analysis of the various fibrinogen chains by SDS PAGE.
DLS analysis.
Antigenic fibrinogen and coagulable fibrinogen activated.

Results

The results obtained are the following:

TABLE 3
Visual inspection and turbidity before and after heating stress

| Stress | Composition | Opalescence | Particles | Turbidity |
|---|---|---|---|---|
| T0 | F1 | V S Op | 0 | 0.037 |
| | F2 | V S Op | 1 | 0.029 |
| | F3 | V S Op | 0 | 0.03 |
| | F4 | V S Op | 1 | 0.028 |
| | F5 | V S Op | 0 | 0.027 |
| | F6 | S Op | 0 | 0.024 |
| | F7 | S Op | 0 | 0.024 |
| | F8 | S Op | 0 | 0.024 |
| | F9 | S Op | 0 | 0.025 |
| | F10 | S Op | 0 | 0.024 |
| | F11 | S Op | 0 | 0.036 |
| | F12 | S Op | 0 | 0.033 |
| | F13 | S Op | 0 | 0.034 |
| Heating stress | F1 | Op | 1 | 0.053 |
| 37° C., T + 7 days | F2 | V S Op | 1 | 0.035 |
| | F3 | V S Op | 1 | 0.034 |
| | F4 | V S Op | 0 | 0.038 |
| | F5 | V S Op | 0 | 0.038 |
| | F6 | S Op | 1 | 0.027 |
| | F7 | S Op | 1 | 0.024 |
| | F8 | S Op | 1 | 0.025 |
| | F9 | S Op | 2 | 0.026 |
| | F10 | S Op | 1 | 0.025 |
| | F11 | S Op | 0 | 0.04 |
| | F12 | S Op | 0 | 0.051 |
| | F13 | S Op | 1 | 0.051 |
| Heating stress | F1 | S Op | 4 | 0.054 |
| 37° C., T + 14 days | F2 | S Op | 1 | 0.035 |
| | F3 | V S Op | 0 | 0.033 |
| | F4 | V S Op | 0 | 0.031 |
| | F5 | V S Op | 0 | 0.031 |
| | F6 | S Op | 1 | 0.047 |
| | F7 | S Op | 0 | 0.045 |
| | F8 | S Op | 1 | 0.043 |
| | F9 | S Op | 1 | 0.042 |
| | F10 | S Op | 1 | 0.046 |
| | F11 | S Op | 1 | 0.034 |
| | F12 | S Op | 1 | 0.035 |
| | F13 | Op | 2 | 0.047 |

Legend:
Opalescence: Op: Opalescent; S Op: slightly Opalescent; V S Op: very slightly Opalescent; V Op: very Opalescent
Particles: 0: particles-free; 1: few particles; 2: particles; 3: many particles; 4: very many particles

Tables 4a and 4b: SDS PAGE results at T0

| | F1 | | F2 | | F3 | | F4 | | F5 | |
|---|---|---|---|---|---|---|---|---|---|---|
| | Molecular | | Molecular | | Molecular | | Molecular | | Molecular | |
| | weight | % | weight | % | weight | % | weight | % | weight | % |
| | kDa/band | intensity | kDa/band | intensity | kDa/band | intensity | kDa/band | intensity | kDa/band | intensity |
| Aα1 | 64.23 | 21.2 | 64.84 | 21.4 | 64.00 | 20.9 | 63.97 | 22.3 | 64.41 | 19.9 |
| Aα2 | 62.52 | 6.1 | 52.46 | 6.9 | 62.45 | 6.8 | 62.41 | 7.3 | 62.84 | 7.1 |
| Aα3 | 60.55 | 9.1 | 66.37 | 8.9 | 66.33 | 8.8 | 60.45 | 8.4 | 60.86 | 8.5 |
| Σα | NA | 36.4 | NA | 37.2 | NA | 36.5 | NA | 38.0 | NA | 35.5 |
| β | 54.06 | 29 | 55.73 | 28.9 | 53.84 | 30.0 | 53.96 | 28.7 | 54.37 | 29.7 |
| γ′ | 49.50 | 4.9 | 49.6 | 3.6 | 49.43 | 4.9 | 49.54 | 5.5 | 49.78 | 5.1 |
| γ | 47.89 | 23.4 | 47.73 | 24.7 | 47.82 | 23.4 | 47.92 | 22.6 | 48.15 | 23.7 |
| Σγ | NA | 28.3 | NA | 28.3 | NA | 28.3 | NA | 28.1 | NA | 28.8 |
| | F6 | | F7 | | F8 | | F9 | | F10 | |
| | Molecular | | Molecular | | Molecular | | Molecular | | Molecular | |
| | weight | % | weight | % | weight | % | weight | % | weight | % |
| | kDa/band | intensity | kDa/band | intensity | kDa/band | intensity | kDa/band | intensity | kDa/band | intensity |
| Aα1 | 64.54 | 22.2 | 63.76 | 23.3 | 63.66 | 23.3 | 63.60 | 20.9 | 63.86 | 23.5 |
| Aα2 | 63.05 | 7.7 | 62.3 | 7.6 | 62.18 | 8.6 | 62.15 | 8.5 | 62.24 | 7.6 |
| Aα3 | 60.58 | 9.3 | 59.94 | 9.3 | 59.96 | 9.7 | 59.8 | 10.5 | 60.05 | 9.8 |
| Σα | — | 39.2 | — | 40.2 | — | 41.6 | — | 39.9 | — | 40.9 |
| β | 54.42 | 30.4 | 53.28 | 29.7 | 53.23 | 29.5 | 53.29 | 30.3 | 53.23 | 29.8 |
| γ′ | 50.01 | 4.7 | 48.95 | 4.1 | 49.05 | 3.8 | 49.23 | 3.8 | 49.03 | 3.6 |
| γ | 48.15 | 25.7 | 47.13 | 25.9 | 47.23 | 25.1 | 47.52 | 26.0 | 47.19 | 25.6 |
| Σγ | — | 30.4 | — | 30.0 | — | 28.9 | — | 29.8 | — | 29.2 |

Tables 5a and 5b: SDS PAGE results at T + 7 days

| | F1 | | F2 | | F3 | | F4 | | F5 | |
|---|---|---|---|---|---|---|---|---|---|---|
| | Molecular | | Molecular | | Molecular | | Molecular | | Molecular | |
| | weight | % | weight | % | weight | % | weight | % | weight | % |
| | kDa/band | intensity | kDa/band | intensity | kDa/band | intensity | kDa/band | intensity | kDa/band | intensity |
| Aα1 | 64.33 | 9.3 | 64.31 | 14.5 | 64.44 | 14.6 | 64.47 | 15.2 | 64.87 | 15.8 |
| Aα2 | 62.49 | 5.1 | 62.61 | 6.7 | 62.74 | 5.8 | 62.71 | 5.7 | 63.14 | 6.1 |
| Aα3 | 60.26 | 17.0 | 60.37 | 14.1 | 60.63 | 13.3 | 60.60 | 12.8 | 61.01 | 11.6 |
| Σα | — | 31.4 | — | 35.3 | — | 33.7 | — | 33.7 | — | 33.5 |
| β | 54.04 | 31.1 | 53.81 | 29.8 | 54.05 | 30.8 | 54.00 | 30.3 | 54.55 | 30.5 |
| γ′ | 49.77 | 6.1 | 49.72 | 4.6 | 49.86 | 5.8 | 49.75 | 5.8 | 49.97 | 5.5 |
| γ | 48.04 | 25.7 | 47.85 | 24.6 | 47.98 | 24.2 | 48.01 | 24.2 | 48.35 | 24.5 |
| Σγ | — | 31.8 | — | 29.2 | — | 30.0 | — | 30.0 | — | 30.0 |
| | F6 | | F7 | | F8 | | F9 | | F10 | |
| | Molecular | | Molecular | | Molecular | | Molecular | | Molecular | |
| | weight | % | weight | % | weight | % | weight | % | weight | % |
| | kDa/band | intensity | kDa/band | intensity | kDa/band | intensity | kDa/band | intensity | kDa/band | intensity |
| Aα1 | 64.37 | 15.3 | 64.11 | 15.1 | 64.35 | 14.7 | 64.12 | 13.9 | 64.22 | 14.2 |
| Aα2 | 62.75 | 5.3 | 62.47 | 5.0 | 62.69 | 6.5 | 62.34 | 5.4 | 62.44 | 5.7 |
| Aα3 | 60.39 | 16.2 | 60.10 | 15.7 | 60.27 | 14.8 | 59.99 | 17.5 | 60.08 | 15.2 |
| Σα | — | 36.8 | — | 35.8 | — | 36.0 | — | 36.8 | — | 35.1 |
| β | 53.93 | 31.7 | 53.42 | 32.9 | 53.53 | 32.3 | 53.32 | 33.0 | 53.26 | 32.4 |
| γ′ | 49.5 | 4.4 | 49.08 | 5.2 | 49.18 | 4.3 | 48.37 | 6.1 | 48.91 | 4.6 |
| γ | 47.71 | 27.1 | 47.20 | 26.1 | 47.35 | 27.5 | 47.27 | 24.1 | 47.22 | 27.9 |
| Σγ | — | 31.5 | — | 31.3 | — | 31.8 | — | 30.2 | — | 32.5 |

Tables 6a and 6b: SDS PAGE results at T + 14 days

| | F1 | | F2 | | F3 | | F4 | | F5 | |
|---|---|---|---|---|---|---|---|---|---|---|
| | Molecular | | Molecular | | Molecular | | Molecular | | Molecular | |
| | weight | % | weight | % | weight | % | weight | % | weight | % |
| | kDa/band | intensity | kDa/band | intensity | kDa/band | intensity | kDa/band | intensity | kDa/band | intensity |
| Aα1 | — | — | 64.42 | 8.2 | 64.47 | 9.1 | 64.52 | 9.3 | 65.07 | 10.9 |
| Aα2 | — | — | 62.49 | 4.2 | 62.53 | 4.5 | 62.74 | 3.8 | 63.26 | 3.7 |
| Aα3 | — | — | 60.32 | 14.7 | 60.2 | 13.8 | 60.54 | 14.2 | 60.88 | 12.5 |
| Σα | — | — | — | 27.1 | — | 27.4 | — | 27.3 | — | 27.1 |
| β | — | — | 53.54 | 33.5 | 53.55 | 34.0 | 53.71 | 33.4 | 54.32 | 34.3 |
| γ′ | — | — | 49.44 | 4.9 | 49.44 | 5.0 | 49.45 | 6.1 | 49.87 | 5.6 |
| γ | — | — | 47.70 | 29.9 | 47.71 | 28.8 | 47.84 | 27.9 | 48.38 | 28.5 |
| Σγ | — | — | — | 34.8 | — | 33.8 | — | 34.0 | — | 34.1 |
| | F6 | | F7 | | F8 | | F9 | | F10 | |
| | Molecular | | Molecular | | Molecular | | Molecular | | Molecular | |
| | weight | % | weight | % | weight | % | weight | % | weight | % |
| | kDa/band | intensity | kDa/band | intensity | kDa/band | intensity | kDa/band | intensity | kDa/band | intensity |
| Aα1 | 64.39 | 6.2 | 64.13 | 7.0 | 64.89 | 7.5 | 64.32 | 6.5 | 64.75 | 4.4 |
| Aα2 | 62.00 | 5.0 | 62.35 | 4.5 | 63.2 | 4.5 | 62.37 | 4.5 | 62.95 | 4.9 |
| Aα3 | 60.24 | 16.0 | 60.10 | 14.9 | 60.92 | 14.1 | 60.02 | 14.1 | 60.73 | 15.1 |
| Σα | — | 27.2 | — | 26.4 | — | 26.1 | — | 25.1 | — | 24.4 |
| β | 53.00 | 34.3 | 53.25 | 35.8 | 54.30 | 35.7 | 53.35 | 35.8 | 53.92 | 35.5 |
| γ′ | 49.7 | 6.5 | 49.07 | 7.3 | 49.89 | 7.3 | 48.85 | 9.1 | 49.66 | 6.9 |
| γ | 47.56 | 31.9 | 47.24 | 30.5 | 48.03 | 30.8 | 47.30 | 30.0 | 47.66 | 33.2 |
| Σγ | — | 38.4 | — | 37.8 | — | 38.1 | — | 39.1 | — | 40.1 |

TABLE 7
DLS results at T0, T + 7 days and T + 14 days

| | T0 | | T + 7 days | | T + 14 days | |
|---|---|---|---|---|---|---|
| | Hydrodynamic | % | Hydrodynamic | % | Hydrodynamic | % |
| | radius (nm) | monomer | radius (nm) | monomer | radius (nm) | monomer |
| F1 | 15.1 | 94.8 | 24.1 | 100 | 11.7 | 50 |
| F2 | 14.9 | 98.9 | 17.8 | 98.4 | 16.7 | 92.2 |
| F3 | 14.6 | 96.8 | 13.6 | 72.3 | 16.5 | 92.6 |
| F4 | 14.9 | 91.7 | 15 | 85.2 | 15 | 85.5 |
| F5 | 15.5 | 95.1 | 15.4 | 71 | 17.7 | 97.7 |
| F6 | 14.5 | 98.1 | 13.4 | 69.4 | 14.5 | 55.1 |
| F7 | 14.4 | 98.1 | 14.3 | 77.4 | 13.2 | 53.6 |
| F8 | 14.2 | 95.9 | 15.4 | 85.1 | 13.7 | 57.5 |
| F9 | 14.7 | 97.5 | 15.3 | 82.1 | 14.4 | 63.6 |
| F10 | 14.5 | 94.1 | 16.2 | 89.6 | 15.5 | 69.8 |
| F11 | 15.5 | 97.2 | 15.1 | 64.8 | 12.1 | 57.6 |
| F12 | 15.0 | 95.1 | 15.2 | 64.4 | 11.7 | 54.2 |
| F13 | 16.3 | 100 | 15.4 | 61.6 | 15.3 | 50.24 |

TABLE 8
Results in terms of antigenic fibrinogen and
in terms of coagulable fibrinogen activity
at T0, T + 7 days and T + 14 days

| | F2 | | | F10 | | | F8 | | |
|---|---|---|---|---|---|---|---|---|---|
| Activity | T0 | T7 d | T14 d | T0 | T7 d | T14 d | T0 | T7 d | T14 d |
| Anti- | 15.2 | 15.8 | 15.3 | 15.5 | 15.4 | 15.7 | 15.2 | 15.4 | 15.1 |
| genic | | | | | | | | | |
| fibrin- | | | | | | | | | |
| ogen | | | | | | | | | |
| Coagu- | 12.3 | 9.2 | 5.1 | 13.6 | 8.3 | 2.5 | 13.2 | 7.8 | 2.6 |
| lable | | | | | | | | | |
| fibrin- | | | | | | | | | |
| ogen | | | | | | | | | |

The results show that formulations of these F1-F13 at pH 7.0 comprising between 10 and 300 mM of arginine and of glutamate, and in particular in equimolar amounts, are stable.

### Example 4: Stability of a Liquid Fibrinogen Composition by Long-Term Stability Test

Fibrinogen Composition

The compositions are the same as those of example 3.

Fibrinogen Composition Stability

The composition is subjected to stability testing at 5° C. under air.

Samples are taken at T0, T+1 month, T+2 months, T+3 months and T+6 months for analyses.

Tables 9 and 10: Comparison of formulations F14
and F15 showing the effect of the surfactant

| F14 | | T0 | T1 M | T2 M | T3 M | T6 M |
|---|---|---|---|---|---|---|
| Visual | | particle | particle | particle | particle | particle |
| aspect | | free, very | free, | free, very | free, very | free, very |
| | | slightly | slightly | slightly | slightly | slightly |
| | | opalescent | opalescent | opalescent* | opalescent | opalescent |
| DLS | Hydrodynamic | 15.2 | 16.8 | 15.7 | 16.8 | 17.2 |
| | radius of | | | | | |
| | the h | | | | | |
| | monomers at | | | | | |
| | 90° (nm) | | | | | |
| | Monomers at | 100 | 100 | 94.4 | 98.9 | 100 |
| | 90° (%) | | | | | |
| Turbidity | | 0.036 | 0.036 | 0.034 | 0.036 | 0.025 |
| (OD 400 nm) | | | | | | |
| pH | | 6.83 | | | | 6.8 |
| Osmolality | mOsmol/kg | 353 | | | | 394 |
| Coagulable | | 14.2 | 14.0 | 14.00 | 13.3 | 10.6 |
| fibrinogen | | | | | | |
| Antigenic | | 15.8 | 15.4 | 14.7 | 15.1 | 13.2 |
| fibrinogen | | | | | | |
| Coag | | 0.9 | 0.91 | 0.95 | 0.88 | 0.8 |
| fibri/Ag | | | | | | |
| fibri ratio | | | | | | |
| SDS PAGE | Aα1 (64 kDa) | 21 | 16.2 | 11.3 | 10.6 | 3.5 |
| | Aα2 (62 kDa) | 8.1 | 6.8 | 3.2 | 2.7 | 3.7 |
| | Aα3 (60 kDa) | 8.3 | 15.3 | 17.2 | 22.4 | 23.5 |
| | ΣAαₙ | 37.4 | 38.3 | 31.7 | 35.7 | 30.7 |
| | Bβ (54 kDa) | 28.5 | 28.1 | 25.7 | 29.1 | 30.5 |
| | γ′ (50 kDa) | 4.6 | 4.7 | 5.7 | 6.9 | 5.6 |
| | γ (48 kDa) | 24.6 | 24.5 | 22.5 | 23.2 | 28.2 |
| | Σγ | 29.2 | 29.2 | 28.2 | 30.1 | 33.8 |
| HPSEC | High | 1.5 | 2.7 | 4.7 | 4.6 | 2.5 |
| | molecular | | | | | |
| | weights | | | | | |
| | Monomers | 98.5 | 97.3 | 92.3 | 90.6 | 94.3 |
| | Low | NA | NA | 3 | 4.8 | 3.2 |
| | molecular | | | | | |
| | weights | | | | | |

| F15 | | T0 | T1 M | T3 M | T6 M |
|---|---|---|---|---|---|
| Visual | | particle | few | few | few |
| aspect | | free, very | particles, | particles, | particles, |
| | | slightly | slightly | slightly | slightly |
| | | opalescent | opalescent | opalescent | opalescent |
| DLS | Hydrodynamic | 13.6 | 13.9 | 13.7 | 13.3 |
| | radius of | | | | |
| | the h | | | | |
| | monomers at | | | | |
| | 90° (nm) | | | | |
| | Monomers at | 72 | 69.2 | 70.1 | 63 |
| | 90° (%) | | | | |
| Turbidity | | 0.031 | 0.039 | 0.035 | 0.025 |
| (OD 400 nm) | | | | | |
| pH | | 6.9 | ND | ND | 7 |
| Osmolality | mOsmol/kg | 400 | ND | ND | 402 |
| Coagulable | | 13.7 | ND | 12.9 | 12 |
| fibrinogen | | | | | |
| Antigenic | | 13.5 | ND | 13.9 | 12.3 |
| fibrinogen | | | | | |
| Coag | | 1 | ND | 0.93 | 0.96 |
| fibri/Ag | | | | | |
| fibri ratio | | | | | |
| SDS PAGE | Aα1 (64 kDa) | 17.4 | 16.7 | 11.8 | 7.2 |
| | Aα2 (62 kDa) | 7.8 | 7.7 | 6.8 | 3 |
| | Aα3 (60 kDa) | 7 | 7.3 | 12.8 | 16.5 |
| | ΣAαₙ | 32.2 | 31.7 | 31.4 | 26.7 |
| | Bβ (54 kDa) | 31.8 | 32.1 | 33.6 | 34.3 |
| | γ′ (50 kDa) | 3.9 | 5.2 | 6.3 | 5.1 |
| | γ (48 kDa) | 28.2 | 27.3 | 25.2 | 30.8 |
| | Σγ | 32.1 | 32.5 | 31.5 | 35.9 |
| HPSEC | High | ND | ND | 3.1 | 3.6 |
| | molecular | | | | |
| | weights | | | | |
| | Monomers | ND | ND | 93.3 | 93.7 |
| | Low | ND | ND | 3.6 | 2.7 |
| | molecular | | | | |
| | weights | | | | |

### Example 5: Stability of a Liquid Fibrinogen Composition by Accelerated Stability Test

Fibrinogen Compositions

The composition obtained in example 1 is formulated with various excipients.

The formulations are reproduced in table 11 below:

| | | | | Amino | Trisodium | | | | |
|---|---|---|---|---|---|---|---|---|---|
| | | Glutamate | Arginine | acid | citrate | | | | |
| Formulation | Fibrinogen | (mM) | (mM) | (mM) | (mM) | Surfactant | Other | pH | Osmolality |
| F16 | 15 g/l, | 80 | 150 | | 8.5 | | | 6.0 | 432 |
| F17 | derived | 80 | 150 | | 8.5 | | | 7.0 | 434 |
| F18 | from | 50 | 100 | | 8.5 | | | 6.0 | 295 |
| F19 | example 1 | 50 | 200 | | 8.5 | | | 6.0 | 457 |
| F20 | | 100 | 100 | | 8.5 | | | 6.0 | 385 |
| F21 | | 100 | 200 | | 8.5 | | | 6.0 | 544 |
| F22 | | 100 | 200 | Serine | 8.5 | Polysorbate | | 6.0 | 518 |
| | | | | 100 | | 80 200 ppm | | | |
| F23 | | 100 | 200 | Serine | 8.5 | Polysorbate | | 7.0 | 530 |
| | | | | 100 | | 80 200 ppm | | | |
| F24 | | 100 | 200 | | 8.5 | Polysorbate | Albumin | 6.0 | 429 |
| | | | | | | 80 200 ppm | 500 ppm | | |
| F25 | | 100 | 200 | Serine | 8.5 | Polysorbate | Albumin | 6.0 | 514 |
| | | | | 100 | | 80 200 ppm | 500 ppm | | |
| F26 | | 100 | 200 | Serine | 8.5 | Polysorbate | Albumin | 6.0 | 516 |
| | | | | 100 | | 80 200 ppm | 500 ppm | | |

Analysis Protocols

The compositions are subjected to a heating stress by heating in a thermostatic chamber at 37° C., then removed from the thermostatic chamber for analysis at T0, T+7 days and T+14 days.

The various analyses carried out are the following:
Visual inspection: opalescence and formation of visible particles are determined by visual inspection carried out with a European pharmacopeia inspecting device
Turbidity
pH
Osmolality
Analysis of the various fibrinogen chains by SDS PAGE
DLS analysis

Results

The results obtained are the following:

Visual inspection:

| Formulation | | T0 | T7 d 37° C. | T14 d 37° C. |
|---|---|---|---|---|
| F16 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F17 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F18 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F19 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F20 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F21 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F22 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F23 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F24 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F25 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |
| F26 | Particles - | Very slightly | Very slightly | Very slightly |
| | Ph. Eur | opalescent | opalescent | opalescent |
| | Opalescence - | Free of | Free of | Free of |
| | Ph. Eur | particle | particle | particle |

Turbidity

| OD 400 nm | T0 | T7 d | T14 d |
|---|---|---|---|
| F16 | 0.021 | 0.024 | 0.030 |
| F17 | 0.018 | 0.022 | 0.033 |
| F18 | 0.021 | 0.034 | 0.048 |
| F19 | 0.016 | 0.023 | 0.033 |
| F20 | 0.021 | 0.029 | 0.036 |
| F21 | 0.018 | 0.022 | 0.032 |
| F22 | 0.019 | 0.028 | 0.031 |
| F23 | 0.019 | 0.024 | 0.030 |
| F24 | 0.019 | 0.031 | 0.032 |
| F25 | 0.019 | 0.031 | 0.032 |
| F26 | 0.017 | 0.022 | 0.027 |

Monitoring of pH

| pH | T0 | T7 d | T14 d |
|---|---|---|---|
| F16 | 6.0 | 6.0 | 6.1 |
| F17 | 7.0 | 7.0 | 7.1 |
| F18 | 6.1 | 6.1 | 6.1 |
| F19 | 6.0 | 6.1 | 6.1 |
| F20 | 6.1 | 6.1 | 6.1 |
| F21 | 6.0 | 6.0 | 6.0 |
| F22 | 6.1 | 6.1 | 6.1 |
| F23 | 7.0 | 6.9 | 7.0 |
| F24 | 6.0 | 6.0 | 6.1 |
| F25 | 6.0 | 6.0 | 6.0 |
| F26 | 6.9 | 6.9 | 6.9 |

Osmolality

| Formulation | T0 | T7 d | T14 d |
|---|---|---|---|
| F16 | 432 | 433 | 431 |
| F17 | 434 | 438 | 435 |
| F18 | 295 | 295 | 294 |
| F19 | 457 | 465 | 462 |
| F20 | 385 | 386 | 385 |
| F21 | 544 | 546 | 545 |
| F22 | 518 | 516 | 515 |
| F23 | 530 | 534 | 526 |
| F24 | 429 | 431 | 429 |
| F25 | 514 | 514 | 516 |
| F26 | 516 | 513 | 512 |

DLS analysis

| Formulation | | T0 | T7 d | T14 d |
|---|---|---|---|---|
| F16 | Rh (nm) | 14.0 | 15.6 | 14.9 |
| | % monomer | 100 | 96.0 | 80.2 |
| | ITD (a.u.) | 19.42 | 23.03 | 25.13 |
| F17 | Rh (nm) | 13.8 | 13.9 | 14.6 |
| | % monomer | 100 | 93.3 | 85.2 |
| | ITD (a.u.) | 19.10 | 21.23 | 23.34 |
| F18 | Rh (nm) | 14.5 | 13.2 | 14.0 |
| | % monomer | 100 | 64.3 | 58.8 |
| | ITD (a.u.) | 24.26 | 32.43 | 38.25 |
| F19 | Rh (nm) | 13.8 | 12.8 | 13.3 |
| | % monomer | 100 | 71.3 | 59.8 |
| | ITD (a.u.) | 20.02 | 24.59 | 29.75 |
| F20 | Rh (nm) | 14.8 | 13.9 | 14.0 |
| | % monomer | 100 | 80.0 | 71.6 |
| | ITD (a.u.) | 23.08 | 27.24 | 29.21 |
| F21 | Rh (nm) | 13.9 | 15.6 | 13.2 |
| | % monomer | 100 | 97.7 | 73.3 |
| | ITD (a.u.) | 19.52 | 22.00 | 24.16 |
| F22 | Rh (nm) | 14.4 | 13.7 | 13.7 |
| | % monomer | 100 | 74.5 | 64.2 |
| | ITD (a.u.) | 19.22 | 25.03 | 28.17 |
| F23 | Rh (nm) | 13.6 | 13.9 | 14.6 |
| | % monomer | 97.9 | 87.3 | 79.9 |
| | ITD (a.u.) | 18.93 | 21.85 | 24.02 |
| F24 | Rh (nm) | 13.6 | 12.8 | 12.0 |
| | % monomer | 98.9 | 74.8 | 56.5 |
| | ITD (a.u.) | 19.63 | 23.85 | 27.65 |
| F25 | Rh (nm) | 14.0 | 13.9 | 13.1 |
| | % monomer | 100 | 83.1 | 66.5 |
| | ITD (a.u.) | 19.25 | 22.98 | 27.06 |
| F26 | Rh (nm) | 13.9 | 15.1 | 14.1 |
| | % monomer | 100 | 100.0 | 83.8 |
| | ITD (a.u.) | 19.09 | 20.61 | 22.31 |

At T0

| | F16 | | F17 | | F18 | | F19 | | F20 | | F21 | |
|---|---|---|---|---|---|---|---|---|---|---|---|---|
| | MW | % | MW | % | MW | % | MW | % | MW | % | MW | % |
| SDS PAGE | kDa/ | inten- | kDa/ | inten- | kDa/ | inten- | kDa/ | inten- | kDa/ | inten- | kDa/ | inten- |
| R | band | sity | band | sity | band | sity | band | sity | band | sity | band | sity |
| Aα1 | 64 | 23.8 | 64 | 24.6 | 63 | 25.1 | 63 | 24.4 | 63 | 25.4 | 63 | 23.5 |
| Aα2 | 63 | 9.2 | 62 | 10.4 | 62 | 9.5 | 62 | 10.6 | 62 | 9.5 | 62 | 10.7 |
| Aα3 | 61 | 6.1 | 60 | 5.4 | 60 | 5.1 | 59 | 6.7 | 60 | 6.8 | 60 | 6.2 |
| Σα | — | 39.1 | — | 40.4 | — | 39.7 | — | 41.7 | — | 41.7 | — | 40.4 |
| β | 54 | 31.9 | 53 | 31.4 | 53 | 31.4 | 53 | 30.9 | 53 | 30.2 | 53 | 31.0 |
| γ′ | 49 | 4.1 | 49 | 4.4 | 48 | 5.6 | 48 | 5.4 | 48 | 5.1 | 48 | 4.2 |
| γ | 48 | 24.8 | 47 | 23.9 | 47 | 23.3 | 47 | 22.0 | 47 | 23.0 | 48 | 24.3 |
| Σγ | — | 28.8 | — | 28.3 | — | 28.9 | — | 27.4 | — | 28.1 | — | 28.5 |

| | F22 | | F23 | | F24 | | F25 | | F26 | |
|---|---|---|---|---|---|---|---|---|---|---|
| | MW | % | MW | % | MW | % | MW | % | MW | |
| SDS PAGE | kDa/ | inten- | kDa/ | inten- | kDa/ | inten- | kDa/ | inten- | kDa/ | % inten- |
| R | band | sity | band | sity | band | sity | band | sity | band | sity |
| Aα1 | 63 | 24.2 | 64 | 24.5 | 65 | 23.5 | 64 | 22.7 | 64 | 23.3 |
| Aα2 | 62 | 9.9 | 62 | 9.6 | 63 | 11.7 | 62 | 13.9 | 63 | 11.7 |
| Aα3 | 60 | 6.9 | 60 | 6.5 | 62 | 9.4 | 60 | 5.0 | 61 | 8.5 |
| Σα | — | 41.0 | — | 40.6 | — | 44.6 | — | 41.6 | — | 43.5 |
| β | 53 | 31.8 | 53 | 31.2 | 55 | 30.7 | 53 | 30.1 | 54 | 29.6 |
| γ′ | 49 | 3.5 | 49 | 4.6 | 49 | 7.8 | 49 | 5.0 | 49 | 5.0 |
| γ | 47 | 23.8 | 47 | 23.6 | 48 | 16.9 | 48 | 23.2 | 48 | 21.8 |
| Σγ | — | 27.3 | — | 28.2 | — | 24.7 | — | 28.2 | — | 26.8 |

At T + 7 d

| | F16 | | F17 | | F18 | | F19 | | F20 | | F21 | |
|---|---|---|---|---|---|---|---|---|---|---|---|---|
| | MW | % | MW | % | MW | % | MW | % | MW | % | MW | % |
| SDS PAGE | kDa/ | inten- | kDa/ | inten- | kDa/ | inten- | kDa/ | inten- | kDa/ | inten- | kDa/ | inten- |
| R | band | sity | band | sity | band | sity | band | sity | band | sity | band | sity |
| Aα1 | 65 | 22.2 | 65 | 18.8 | 65 | 19.0 | 65 | 21.6 | 65 | 19.9 | 65 | 22.0 |
| Aα2 | 63 | 8.5 | 63 | 8.6 | 63 | 7.1 | 63 | 10.2 | 63 | 9.8 | 63 | 9.1 |
| Aα3 | 61 | 7.3 | 61 | 9.4 | 61 | 9.3 | 61 | 7.6 | 61 | 7.3 | 61 | 7.2 |
| Σα | — | 38.0 | — | 36.8 | — | 35.4 | — | 39.4 | — | 37.0 | — | 38.3 |
| β | 54 | 32.3 | 54 | 33.6 | 54 | 33.2 | 54 | 31.4 | 54 | 33.2 | 54 | 32.8 |
| γ′ | 49 | 5.4 | 50 | 2.6 | 49 | 5.9 | 50 | 4.6 | 49 | 7.0 | 50 | 3.9 |
| γ | 48 | 24.3 | 48 | 26.9 | 48 | 25.5 | 48 | 24.5 | 48 | 22.8 | 48 | 25.0 |
| Σγ | — | 29.7 | — | 29.5 | — | 31.4 | — | 29.1 | — | 29.8 | — | 28.9 |

| | F22 | | F23 | | F24 | | F25 | | F26 | |
|---|---|---|---|---|---|---|---|---|---|---|
| | MW | % | MW | % | MW | % | MW | % | MW | % |
| SDS PAGE | kDa/ | inten- | kDa/ | inten- | kDa/ | inten- | kDa/ | inten- | kDa/ | inten- |
| R | band | sity | band | sity | band | sity | band | sity | band | sity |
| Aα1 | 65 | 24.0 | 65 | 19.6 | 65 | 21.0 | 65 | 19.2 | 65 | 18.4 |
| Aα2 | 63 | 9.5 | 64 | 7.8 | 63 | 11.6 | 63 | 13.3 | 64 | 14.2 |
| Aα3 | 61 | 8.7 | 61 | 9.0 | 62 | 10.2 | 62 | 9.5 | 62 | 9.3 |
| Σα | — | 42.2 | — | 36.4 | — | 42.8 | — | 42.0 | — | 41.9 |
| β | 54 | 35.8 | 55 | 33.4 | 55 | 30.7 | 55 | 30.5 | 55 | 30.7 |
| γ′ | 50 | 4.8 | 50 | 6.0 | 51 | 1.9 | 50 | 3.1 | 50 | 4.2 |
| γ | 48 | 17.2 | 49 | 24.3 | 49 | 24.7 | 49 | 24.5 | 49 | 23.2 |
| Σγ | — | 22.0 | — | 30.3 | — | 26.6 | — | 27.6 | — | 27.4 |

At T + 14 d

| | F16 | | F17 | | F18 | | F19 | | F20 | | F21 | |
|---|---|---|---|---|---|---|---|---|---|---|---|---|
| | MW | % | MW | % | MW | % | MW | % | MW | % | MW | % |
| SDS PAGE | kDa/ | inten- | kDa/ | inten- | kDa/ | inten- | kDa/ | inten- | kDa/ | inten- | kDa/ | inten- |
| R | band | sity | band | sity | band | sity | band | sity | band | sity | band | sity |
| Aα1 | 65 | 16.5 | 65 | 15.5 | 65 | 11.8 | 65 | 19.0 | 65 | 14.7 | 65 | 18.1 |
| Aα2 | 64 | 8.4 | 64 | 6.7 | 63 | 8.7 | 64 | 9.5 | 63 | 9.7 | 63 | 9.0 |
| Aα3 | 62 | 8.5 | 61 | 11.4 | 61 | 10.7 | 61 | 8.6 | 61 | 9.7 | 61 | 8.5 |
| Σα | — | 33.4 | — | 33.6 | — | 31.2 | — | 37.1 | — | 34.1 | — | 35.6 |
| β | 55 | 33.9 | 55 | 33.8 | 55 | 34.0 | 55 | 31.8 | 54 | 32.9 | 54 | 31.8 |
| γ′ | 50 | 5.5 | 50 | 5.5 | 50 | 6.4 | 50 | 6.4 | 49 | 7.7 | 50 | 5.3 |
| γ | 49 | 27.2 | 49 | 27.1 | 49 | 28.5 | 49 | 24.7 | 48 | 25.2 | 48 | 27.2 |
| Σγ | — | 32.7 | — | 32.6 | — | 34.9 | — | 31.1 | — | 32.9 | — | 32.5 |

| | F22 | | F23 | | F24 | | F25 | | F26 | |
|---|---|---|---|---|---|---|---|---|---|---|
| | MW | % | MW | % | MW | % | MW | % | MW | % |
| SDS PAGE | kDa/ | inten- | kDa/ | inten- | kDa/ | inten- | kDa/ | inten- | kDa/ | inten- |
| R | band | sity | band | sity | band | sity | band | sity | band | sity |
| Aα1 | 65 | 16.0 | 65 | 15.5 | 65 | 14.9 | 65 | 14.1 | 65 | 13.4 |
| Aα2 | 63 | 8.9 | 64 | 5.8 | 64 | 14.3 | 63 | 14.4 | 64 | 13.7 |
| Aα3 | 61 | 8.1 | 61 | 10.5 | 62 | 9 | 62 | 9.3 | 62 | 11.8 |
| Σα | — | 33.0 | — | 31.8 | — | 39.2 | — | 37.8 | — | 38.9 |
| β | 55 | 33.2 | 55 | 34.5 | 55 | 31.2 | 55 | 32.0 | 55 | 29.9 |
| γ′ | 50 | 7.1 | 50 | 5.1 | 50 | 6.8 | 51 | 2.2 | 51 | 3.8 |
| γ | 48 | 26.7 | 49 | 28.6 | 49 | 23.7 | 49 | 28.1 | 49 | 27.4 |
| Σγ | — | 33.8 | — | 33.7 | — | 30.5 | — | 30.3 | — | 31.2 |

The results show that formulations F16-F26 at pH 6.0-8.0, comprising between 50 and 200 mM of arginine and of glutamate, are stable.

### Example 6: Stability of a Liquid Fibrinogen Composition by Long-Term Stability Test

Fibrinogen Compositions

Composition obtained in example 2 is formulated with various excipients.

The formulations are reproduced in table 11 below:

| | | | | | | | | | |
|---|---|---|---|---|---|---|---|---|---|
| | | | | Amino | Trisodium | | | | |
| | | Glutamate | Arginine | acid | citrate | | | | |
| Formulation | Fibrinogen | (mM) | (mM) | (mM) | (mM) | Surfactant | Other | pH | Osmolality |

| | | | | | | | | |
|---|---|---|---|---|---|---|---|---|
| F27 | 15 g/l, | 80 | 150 | Serine | 8.5 | Polysorbate | 6.0 | 533 |
| | derived | | | 100 | | 80 200 ppm | | |
| F28 | from | 80 | 150 | Serine | 8.5 | Polysorbate | 7.0 | 529 |
| | example 2 | | | 100 | | 80 200 ppm | | |

Analysis Protocols

The compositions are stored in a thermostatic chamber at 5° C., 25° C. and 37° C., then removed from the thermostatic chamber for analysis at T0, T+14 days and T+1 month.

The various analyses carried out are the following:
Visual inspection: opalescence and formation of visible particles are determined by visual inspection carried out with a European pharmacopeia inspecting device.
Turbidity
pH
Osmolality
DLS analysis
Coagulable fibrinogen and antigenic fibrinogen

Analysis of the various fibrinogen chains by SDS PAGE.

Results

The results obtained are the following:

Visual inspection:

| | | T0 | | T14 d | | T1 M | |
|---|---|---|---|---|---|---|---|
| | | Particle | Opalescence | Particle | Opalescence | Particle | Opalescence |
| | | Ph. Eur. | Ph. Eur. | Ph. Eur. | Ph. Eur. | Ph. Eur. | Ph. Eur. |
| F27 | | | | | | | |
| Visual | 5° C. | O | S op | | | | |
| inspection | 25° C. | | | | | O | V S op |
| | 37° C. | | | O | S op | O | S op |
| F28 | | | | | | | |
| Visual | 5° C. | O | S op | | | | |
| inspection | 25° C. | | | | | O | V S op |
| | 37° C. | | | O | S op | O | S op |

Turbidity

| | | | T0 | T14 d | T1 M |
|---|---|---|---|---|---|
| F27 | | | | | |
| | Turbidity | 5° C. | 0.008 | | |
| | | 25° C. | | | 0.026 |
| | | 37° C. | | 0.046 | 0.055 |
| F28 | | | | | |
| | Turbidity | 5° C. | 0.011 | | |
| | | 25° C. | | | 0.027 |
| | | 37° C. | | 0.035 | 0.043 |

pH and osmolality

| | | | T0 | T14 d | T1 M |
|---|---|---|---|---|---|
| F27 | | | | | |
| | pH | 5° C. | 6.1 | | |
| | | 25° C. | | | |
| | | 37° C. | | 6.1 | 6.1 |
| | Osmolality | 5° C. | 533 | | |
| | | 25° C. | | | 534 |
| | | 37° C. | | 534 | 536 |
| F28 | | | | | |
| | pH | 5° C. | 6.9 | | |
| | | 25° C. | | | 6.9 |
| | | 37° C. | | 6.9 | 6.9 |
| | Osmolality | 5° C. | 529 | | |
| | | 25° C. | | | 534 |
| | | 37° C. | | 534 | 533 |

DLS analysis

| | | T0 | | T14 d | | T1 M | |
|---|---|---|---|---|---|---|---|
| | | Monomer | ITD at 90° | Monomer | ITD at 90° | Monomer | ITD at 90° |
| | | at 90° (%) | (a.u.) | at 90° (%) | (a.u.) | at 90° (%) | (a.u.) |
| F27 | | | | | | | |
| DLS | 5° C. | 100 | 25.43 | | | | |
| | 25° C. | | | | | 96.2 | 28.89 |
| | 37° C. | | | 38.9 | 56.27 | 30.7 | 71.90 |
| F28 | | | | | | | |
| DLS | 5° C. | 98.4 | 25.15 | | | | |
| | 25° C. | | | | | 90.4 | 28.24 |
| | 37° C. | | | 46.9 | 48.07 | 37.5 | 57.38 |

Coagulable fibrinogen and antigenic fibrinogen

| | | | F27 | F28 |
|---|---|---|---|---|
| Stability at +5° C. | | | | |
| | T0 | Fg activity (g/l) | 15.7 | 15.9 |
| | | Antigenic Fg | 15.7 | 15.2 |
| | | Ratio | 1.0 | 1.0 |
| Stability at +25° C. | | | | |
| | T0 | Fg activity (g/l) | 15.7 | 15.9 |
| | | Antigenic Fg | 15.7 | 15.2 |
| | | Ratio | 1.0 | 1.0 |
| | T1 M | Fg activity | 14.3 | 12.7 |
| | | Antigenic Fg | 16.0 | 15.2 |
| | | Ratio | 0.9 | 0.8 |

Analysis of the various fibrinogen chains
by SDS PAGE under reducing conditions

| mW (Kda) | T 0 | T14 d 37° C. | T1 M 25° C. | T1 M 37° C. |
|---|---|---|---|---|
| F27 | | | | |
| 219 | 2.1 | 2.4 | 2.6 | 2.2 |
| 153 | 0.9 | 1.1 | 0.7 | 0.8 |
| 101 | 1.7 | 1.4 | 1.3 | 1.5 |
| 64 Aα1 | 23.2 | 16.6 | 20.0 | 16.0 |
| 62 Aα2 | 9.7 | 9 | 7.5 | 7.5 |
| 60 Aα3 | 2 | 3.6 | 3.3 | 4.4 |
| Σ64/62/60 | 34.9 | 29.2 | 30.8 | 27.9 |
| 54 Bβ | 31.4 | 34.1 | 33.3 | 34.2 |
| 50 γ′ | 2.9 | 3.1 | 2.8 | 2.7 |
| 48γ | 25.7 | 27.6 | 28.0 | 30.0 |
| Σ50/48 | 28.6 | 30.7 | 30.8 | 32.7 |
| 34 | 0.3 | 1 | 0.4 | 0.7 |
| 29 | nd | nd | nd | nd |
| F28 | | | | |
| 219 | 2.8 | 2.3 | 2.4 | 2.5 |
| 153 | 0.8 | 0.8 | 1.0 | 0.9 |
| 101 | 1.8 | 1.8 | 1.7 | 2.1 |
| 64 Aα1 | 23.7 | 16 | 12.5 | 9.4 |
| 62 Aα2 | 8.2 | 7.8 | 8.1 | 7.8 |
| 60 Aα3 | 2.0 | 4.0 | 3.0 | 4.8 |
| Σ64/62/60 | 33.9 | 27.8 | 23.6 | 22.0 |
| 54 Bβ | 31.4 | 33.9 | 35.0 | 35.1 |
| 50 γ′ | 2.3 | 2.7 | 3.2 | 6.6 |
| 48γ | 26.6 | 29.6 | 30.5 | 29.8 |
| Σ50/48 | 28.9 | 32.3 | 33.7 | 36.4 |
| 34 | 0.6 | 1.2 | 1.0 | 0.8 |
| 29 | nd | nd | 1.5 | 1.2 |

The results show that formulations F27-F28 at pH 6.0-7.0, comprising 80 mM of glutamate and 150 mM of arginine, in the presence of surfactant, of buffer and of a supplementary amino acid, are stable.

1.-24. (canceled)

25. A liquid pharmaceutical composition, comprising:
human fibrinogen,
between 10 and 300 mM arginine, and
between 10 and 300 mM glutamate;
wherein the pH of the composition is between 6 and 8.

26. The pharmaceutical composition according to claim 25, wherein the arginine concentration and the glutamate concentration are between 10 and 300 mM, between 20 and 200 mM, between 30 and 100 mM, or between 50 and 80 mM.

27. The pharmaceutical composition according to claim 25, wherein the arginine concentration and the glutamate concentration are between 10 and 80 mM, between 20 and 70 mM, or between 30 and 60 mM.

28. The pharmaceutical composition according to claim 25, wherein the glutamate concentration is less than 80 mM, less than 70 mM, less than 60 mM, or less than 50 mM.

29. The pharmaceutical composition according to claim 25, wherein the glutamate concentration is between 10 and 80 mM, between 20 and 70 mM, or between 30 and 60 mM.

30. The pharmaceutical composition according to claim 25, wherein the arginine concentration is between 10 and 300 mM, between 20 and 250 mM, or between 50 and 250 mM.

31. The pharmaceutical composition according to claim 25, wherein the arginine and the glutamate are present in equimolar amounts at a ratio of between 0.8 and 1.2, between 0.9 and 1.1, or approximately equal to 1.0.

32. The pharmaceutical composition according to claim 25, wherein the fibrinogen concentration is between 10 and 30 g/l, between 15 and 25 g/l, or between 15 and 20 g/1.

33. The pharmaceutical composition according to claim 25, further comprising at least one amino acid selected from alanine, asparagine, aspartate, cysteine, glutamine, glycine, histidine, isoleucine, leucine, lysine, methionine, phenylalanine, proline, serine, threonine, tryptophan, tyrosine, or valine, or a combination thereof.

34. The pharmaceutical composition according to claim 25, wherein further comprising at least one hydrophobic amino acid selected from leucine, alanine, phenylalanine, tryptophan, valine, methionine, isoleucine, proline, cysteine, and glycine, or a combination thereof.

35. The pharmaceutical composition according to claim 34, wherein the amino acid is proline, isoleucine, or serine, or a combination thereof.

36. The pharmaceutical composition according to claim 33, wherein the amino acid concentration is between 1 and 300 mM, between 10 and 200 mM, or between 20 and 100 mM.

37. The pharmaceutical composition according to claim 25, further comprising a surfactant, preferentially polysorbate 80 or polysorbate 20 or Pluronic®F68.

38. The pharmaceutical composition according to claim 37, wherein the surfactant concentration is between 1 and 500 ppm, between 50 and 400 ppm, or between 150 and 250 ppm.

39. The pharmaceutical composition according to claim 25, further comprising a buffer.

40. The pharmaceutical composition according to claim 39, wherein the buffer comprises trisodium citrate.

41. The pharmaceutical composition according to claim 40, wherein the trisodium citrate concentration is between 1 and 15 mM or is between 7 and 10 mM.

42. The pharmaceutical composition according to claim 25, wherein the composition has pH between 6 and 7.5 or is between 6 and 7.

43. The pharmaceutical composition according to claim 25, wherein the composition is devoid of sodium chloride and/or albumin.

44. The pharmaceutical composition according to claim 25, wherein the composition has an osmolality of between 250 and 650 mOsm/kg, or between 300 and 550 mOsm/kg.

45. The liquid pharmaceutical composition according to claim 25, comprising:
10-30 g/l fibrinogen,
10-300 mM arginine,
10-300 mM glutamate,
optionally 1-15 mM buffer, in particular of trisodium citrate,
optionally 1-300 mM of another amino acid, in particular 1-300 mM of proline and/or 1-300 mM of isoleucine and/or 1-300 mM of serine,
optionally 1-500 ppm, 20-400 ppm, or 150-250 ppm surfactant, and
optionally 5-1000 ppm human albumin;
wherein the composition has pH 6.0-8.0.

46. The liquid pharmaceutical composition according to claim 25, comprising:
10-30 g/l fibrinogen,
20-200 mM arginine,
20-200 mM glutamate,
optionally 7-10 mM of buffer, in particular of trisodium citrate,
optionally 1-100 mM of another amino acid, in particular 1-100 mM of proline and/or 1-100 mM of isoleucine and/or 1-100 mM of serine,
optionally 20-400 ppm surfactant, and
optionally 5-500 ppm human albumin;
wherein the composition has pH 6.0-8.0.

47. The liquid pharmaceutical composition according to claim 25, further comprising:
15-25 g/l fibrinogen,
150 mM arginine,
80 mM glutamate,
8.5 mM trisodium citrate,
200 ppm surfactant, wherein the surfactant is optionally polysorbate 80, polysorbate 20, or Pluronic®F68,
optionally 1-100 mM of another amino acid, wherein the other amino acid is optionally proline, isoleucine, or 1-100 mM of serine, and
optionally 5-1000 ppm of human albumin;
wherein composition has pH 6.0-7.0.

48. The pharmaceutical composition according to claim 25, wherein the fibrinogen is obtained by blood plasma fractionation.
